(19) United States
(12) Patent Application Publication
(10) Pub. No.: US 2009/0062391 A1
(43) Pub. Date: Mar. 5, 2009

(54) Use of a virucidal ointment in the nares for prevention of transmission and contraction of common colds

(75) Inventor: Kent Christopher New, Ponte Vedra Beach, FL (US)

Correspondence Address: KENT C. NEW, 104 KINGFISHER DRIVE, PONTE VEDRA BEACH, FL, 32082, US

(73) Assignee: Kent Christopher New, Ponte Vedra Beach, FL (US)

(21) Appl. No.: 11/845,756

(22) Filed: Aug. 27, 2007

(51) Int. Cl. A61K 31/19; A61K 31/194; A61K 31/195; A61K 47/00; A61K 47/10; A61K 47/44; A61P 31/16

(52) U.S. Cl. 514/561; 514/557; 514/574; 514/772; 514/782

(57) ABSTRACT

A novel method of preventing or inhibiting the contraction or communication of viruses which cause the common cold is proposed. Application of a virucidal ointment containing one or more organic acids (e.g. malic acid) with or without a surfactant (e.g. sodium C.sub.14-C.sub.16 olefin sulfonate) in a carrier agent (e.g. petrolatum) to the nares of well individuals will reduce the chance that they contract illness with the viruses which cause colds. Application of said ointment to the nares of individuals with a cold will reduce the chance they transmit the illness by reducing self-contamination of their hands when touching their nose. The ointment is most effective when applied with a clean applicator multiple times daily.

### CROSS-REFERENCE TO RELATED APPLICATIONS

Not Applicable

### STATEMENT REGARDING FEDERALLY SPONSORED RESEARCH OR DEVELOPMENT

No federally sponsored research was used in the development of this invention.

### REFERENCE TO SEQUENCE LISTING, A TABLE, OR A COMPACT DISC APPENDIX

Not applicable.

### BACKGROUND OF THE INVENTION

1. Field of the Invention

The present invention relates to virucidal compositions for the prevention of the spread of the common cold. Specifically, this invention relates to the use of a virucidal ointment in the nares of uninfected individuals to reduce the chance that they become infected with viruses such as rhinovirus, adenovirus, and parainfluenza virus which are the primary causative agents of illnesses known in lay terms as the “common cold.” The invention may also be used in the nares of infected individuals to reduce transmission of viruses which cause the common cold.

The term “common cold” refers to upper respiratory illnesses caused by infection with one of a variety of viruses. Viruses which cause common colds include: rhinovirus, coronavirus, adenovirus, respiratory syncytial virus, enterovirus, and parainfluenza [1]. Infection with these viruses leads to a self limited illness generally lasting several days to up to two weeks. The associated morbidity of these infections is responsible for approximately $15 billion in direct health care costs, and about $9 billion in indirect costs [2].

Although certain medications have been demonstrated to shorten the duration of common colds (e.g. zinc), there is no cure for these illnesses. Rhinovirus alone has over 100 known serotypes, and the other viruses which cause colds have many serotypes as well. The vast array of viruses and serotypes which can cause a cold makes the development of an effective vaccine highly unlikely. The focus of efforts to reduce the impact of the common cold, therefore, has centered on the prevention of transmission of the disease.

The spread of common colds frequently occurs through hand-to-hand transmission of virus followed by self contamination with virus of the nasal or conjunctival mucosa [3]. Typically, an individual with a cold will either directly or indirectly contaminate the hands of another individual who does not have the illness. The uninfected individual will thereby have viral particles on their hands. These viral particles initiate infection after the individual contaminates their own nasal or conjunctival mucosa with the viral particles on their hands. In this way, although transmission occurs through the initial step of hand-to-hand transfer of viral particles, infection of new individuals typically occurs only if the viral particles gain access to the nasal or conjunctival mucosa.

Knowledge of the primary causative agents and the mechanism of transmission of common colds have been in the public domain for over twenty years. Although considerable progress has been made in understanding the molecular biology of viruses responsible for these illnesses, little progress has been made in the prevention or treatment of colds. Efforts aimed at inactivating offending viral particles have focused on cleaning inanimate objects or developing products for use on the hands. These efforts fail largely due to two reasons: 1, the viruses responsible for colds are difficult to inactivate; and 2, it is nearly impossible to keep one's hands free of viral contamination because they repeatedly come in contact with contaminated objects.

The present invention aims to significantly reduce the spread of common colds. The invention relies on the application of a simple virucidal composition known effective against the viruses which cause colds via a novel method.

2. Description of the Prior Art

The prior art in the prevention of transmission of common colds is extensive, but it has heretofore focused primarily on the inactivation of viral particles on the hands. Previous work lends insight into potential effective agents for use in the current invention.

Hendley et al. investigated the antiviral activity of several compounds including iodine, ethyl alcohol, benzalkonium chloride (BAK), and hexachlorophene [4]. These agents were known for their anti-bacterial activity, but all except iodine performed poorly as antiviral agents. Unfortunately iodine is too irritating to the skin to be frequently applied.

Poli et al. investigated the antiviral activity of several organic acids such as citric, malic, pyruvic, and succinic acids and found them to be effective against herpes simplex virus, rhabdovirus, and other enveloped viruses, but not effective against adenovirus, the only naked virus tested [5]. Hayden, et al. reported that hand lotions containing 2% glutaric acid were more effective than placebo at inactivating certain serotypes of rhinovirus [6]. Rhinoviruses are responsible for more colds than any other virus family, but not all colds. Therefore the use of organic acids alone as an antiviral agent will likely have some effectiveness at reducing the transmission of common cold viruses, but will not offer comprehensive protection.

Snipes, et al. discovered that alcohols of certain chain lengths were effective at inactivating lipid-containing viruses [7]. Unfortunately, the alcohols found most effective are extremely insoluble in aqueous media, making them somewhat difficult to employ in practical applications. However, further work with alcohols has lead to other useful antiviral inventions by Hendley, et al. and Konowalchuk, et al., described below.

Hendley, et al. (U.S. Pat. No. 6,034,133) disclose a virucidal hand lotion containing malic acid, citric acid, and a C1 to C6 alcohol which effectively inactivates rhinovirus contamination of treated hands. Their work also revealed that the use of lotions containing malic acid and citric acid without an alcohol would reduce, but not eliminate rhinovirus contamination of treated hands.

Recently, Konowalchuk, et al. (U.S. Pat. No. 7,045,548) disclose a method of inactivating viruses with a composition of a C1 to C3 monohydroxy alcohol or a C2 to C4 diol with a sufficient amount of an acid (either organic or inorganic) to adjust the pH of the composition to below 4.6. This invention is demonstrated effective in topically treating lesions caused by herpes simplex virus. It is also proposed that the composition may be effective at ameliorating the symptoms of a common cold with intranasal application. No mention is made of the use of the composition in the nares of individuals to reduce transmission or contraction of viral infection.

Brown-Skrobot, et al. (U.S. Pat. No. 4,975,217) disclose a method of inactivating viruses and bacteria through the application of a combination of an organic acid and an anionic surfactant, with or without an alcohol to the hands. This work describes the finding that a combination of an organic acid with an anionic surfactant has antiviral activity against both enveloped and naked viruses, thus is effective against a broad array of viruses able to cause common colds. The invention is described as a virucidal lotion for the hands. There is no mention of the use of the combination of agents in the nares to reduce the transmission or contraction of common colds.

Hossain, et al. (U.S. Pat. No. 4,897,304) disclose the use of a combination of organic acids and a surfactant in a facial tissue to reduce the spread of viruses which cause colds. However, Hayden, et al. reported that the use of facial tissues can interrupt the transmission of viruses whether treated with the antiviral composition or not [8]. Hence no distinct advantage in reducing transmission of viruses which cause the common cold is obtained by this product.

In summary, the prior art contains several examples of various antiviral compositions for use on the hands to reduce transmission of viruses which cause colds. Some of these inventions also disclose use of antiviral compositions in the nasal passages to ameliorate the symptoms of a cold. No prior invention describes the use of an antiviral composition in the nares of individuals to reduce or inhibit transmission or contraction of illness with viruses which cause common colds.

### REFERENCES

### 1. U.S. Patent Documents

| | | |
|---|---|---|
| 4897304 | January 1990 | Hossain, et al |
| 4975217 | December 1990 | Brown-Skrobot, et al |
| 6034133 | March 2000 | Hendley, et al |
| 7045548 | May 2006 | Konowalchuk, et al |

### 2. Other References

1. Spector, S L. The common cold: current therapy and natural history. J. Allergy Clin. Immunol. 95:1133-38, 1995.

2. Lorber, B. The common cold. J. Gen. Intern. Med. 11:229-236, 1996.

3. Gwaltney, J M Jr., et al. Hand-to hand transmission of rhinovirus colds. Ann Intern Med 88(4):463-7, 1978.

4. Hendley, J O, et al. Evaluation of virucidal compounds for inactivation of rhinovirus on hands. Antimicrob. Agents Chemother. 14:690-4, 1978.

5. Poli, G, et al. Virucidal activity of organic acids. Food Chem. (England) 4(4):251-8, 1979.

6. Hayden, G F, et al. Inactivation of rhinovirus on human fingers by virucidal activity of glutaric acid. Antimicrob. Agents. Chemother. 26(6):928-9, 1984.

7. Snipes, W, et al. Inactivation of lipid-containing viruses by long-chain alcohols. Antimicrob. Agents Chemother. 11(1):98-104, 1977.

8. Hayden, G F, et al. The effect of placebo and virucidal paper handkerchiefs on viral contamination of the hand and transmission of experimental rhinoviral infection. J. Infect. Dis. 152(2):403-7, 1985.

### BRIEF SUMMARY OF THE INVENTION

This application proposes a novel method of reducing transmission of viruses which cause the common cold. As opposed to the plethora of products which attempt to keep hands free of viral contaminant, the product of this invention aims at inactivating viruses in the nares. Since contraction of a cold frequently occurs following contamination of the nasal mucosa with virus, the proposed invention is likely to be very effective at reducing contraction and transmission of common colds.

This invention describes a virucidal ointment for application in the nares. The proposed ointment contains a mixture of organic acids, with or without a surfactant in a carrier such as petrolatum or glycerin. The virucidal ointment proposed may be used in well individuals to reduce the chance that they will contract infection with one of the many viruses which can cause a cold. The ointment may also be used by individuals with a cold to reduce the chance they spread the illness to others by reducing self-contamination of their hands from touching their nose.

### BRIEF DESCRIPTION OF THE SEVERAL VIEWS OF THE DRAWING

Not Applicable.

### DETAILED DESCRIPTION OF THE INVENTION

Treating the nares with a virucidal ointment or lotion will reduce the transmission or contraction of viruses which cause the common cold. Since contamination of the nasal mucosa with virus is a common mechanism of contracting a cold, treating the nares with a virucidal ointment a few times a day may significantly reduce the chance an individual contracts a cold.

The invention described may be used in well individuals to reduce the chance that they will contract infection with one of the viruses which cause common colds. Alternatively, the invention may be utilized in infected individuals to reduce the chance they contaminate their hands with virus when touching their nose, thereby reducing the chance they spread the illness.

The invention is described below with preferred embodiments; however it should be understood that it is not intended to limit the invention to the embodiments described. The invention is intended to include all alternatives as may be included within the spirit and scope of the invention as defined by the appended claims.

Organic acids which are effective against viruses which cause colds and are therefore appropriate for use in the invention include: valeric acid, lactic acid, glycolic acid, pelargonic acid, aspartic acid, malic acid, and citric acid. Appropriate embodiments of the invention include any combination of one or more of these organic acids: One embodiment of the invention is malic acid (2% w/v) plus citric acid (2% w/v) in petrolatum. This combination of acids has been demonstrated effective against certain serotypes of rhinovirus. Said ointment would be applied either directly using a finger, or indirectly using an applicator to the nares one or more times per day. Ideally the ointment is to be applied to the nares using a clean applicator several times daily.

The ointment may contain up to 10% w/v of each organic acid, but typically would consist of 2-7% w/v. This ointment can be produced by making concentrated solutions of the desired acids in water and then diluting in the carrier to the appropriate desired final concentration. Potential carriers include: petrolatum, glycerin, xanthan gum, mineral oil, dipropylene glycol, and other non-toxic gels, creams, or ointments.

Organic acids alone have been demonstrated to effectively inactivate only some rhinovirus serotypes, and are ineffective at inactivating adenovirus. However, the combination of organic acids and surfactants, particularly alkyl sulfonates, has shown effectiveness at inactivating viruses from several families including: rhinovirus, adenovirus, and parainfluenza virus. Members of these virus families are responsible for a majority of common colds in adults. Therefore, a much more effective embodiment of the invention consists of a combination of one or more organic acids and one or more surfactants.

A very potent virucidal embodiment of the invention is malic acid (2% w/v) and sodium C.sub.14-C.sub.16 olefin sulfonate (BIO-TERGE AS-40, 0.5% v/v) in petrolatum. This product can be produced by making a concentrated solution of malic acid in water and then diluting appropriately to the desired final concentration with the surfactant and petrolatum. Sodium C.sub.14-C.sub.16 olefin sulfonate is supplied as a 40% solution and therefore is diluted 1:80 to give the final desired concentration of 0.5% v/v.

Other surfactants with demonstrated effectiveness at inactivating cold viruses when combined with organic acids, and therefore those which are suitable for the invention include: sodium dodecyl benzene sulfonate (BIOSOFT D-35X), sodium dodecyl sulfate (SDS), sodium lauryl sulfate (SLS), and sodium dioctyl sulfosuccinate (AEROSOL OT). Appropriate concentration of the organic acid is 0.1-10% w/v, and of the surfactant is 0.1-10% v/v. Most effective and non-irritating embodiments include the organic acid at 2-7% w/v and the surfactant at 0.1-5% v/v.

Appropriate embodiments of the invention include any combination of one or more of the organic acids listed above with one of the surfactants listed in one of the carrier agents listed. Combining more than two of the organic acids is likely to be irritating to the skin making the product less useful, however.

The ointment described in this invention would best be supplied in small plastic tubes with a cone or dome top containing one or more small holes and a cover. To use the ointment, an individual would remove the cover, wipe the top with a clean tissue, squeeze to extrude some of the ointment from the tube onto the surface of the top, and then wipe the top within their nares bilaterally. To reduce chance of viral transmission, individuals should not share tubes of ointment.

1. A method of preventing or inhibiting the contraction or communication of a common cold, said method comprising applying a virucidal ointment to one or both of a subject's nares; the ointment comprising one or more organic acids and one or more carrier agents.

2. The method of claim 1 wherein the ointment is applied to the nares one to multiple times a day.

3. The method of claim 1 wherein said organic acid is selected from the group consisting of valeric acid, lactic acid, glycolic acid, pelargonic acid, aspartic acid, malic acid, and citric acid.

4. The method of claim 1 wherein said carrier agent is selected from the group consisting of petrolatum, glycerin, mineral oil, dipropylene glycol, and xanthan gum.

5. The method of claim 1 wherein the virucidal ointment contains a combination of one or more organic acids, one or more surfactants, and one or more carrier agents, and said ointment is applied to the nares one to multiple times a day.

6. The method of claim 5 wherein said organic acid is selected from the group consisting of valeric acid, lactic acid, glycolic acid, pelargonic acid, aspartic acid, malic acid, and citric acid.

7. The method of claim 5 wherein said surfactant is selected from the group consisting of sodium dodecyl sulfate, sodium lauryl sulfate, sodium dodecyl benzene sulfonate, sodium C.sub.14-C.sub.16 olefin sulfonate, and sodium dioctyl sulfosuccinate.

8. The method of claim 5 wherein said carrier agent is selected from the group consisting of petrolatum, glycerin, mineral oil, dipropylene glycol, and xanthan gum.

9. The method of claim 5 wherein the virucidal ointment contains malic acid at a concentration of 2% w/v and sodium C.sub.14-C.sub.16 olefin sulfonate at a concentration of 0.5% v/v in petrolatum.

10. The method of claim 5 wherein the virucidal ointment contains malic acid at a concentration of 2% w/v, citric acid at a concentration of 2% w/v, and sodium C.sub.14-C.sub.16 olefin sulfonate at a concentration of 0.5% v/v in petrolatum.

11. The method of claim 5 wherein the virucidal ointment contains one or more organic acids at a concentration between 0.1% and 10% w/v and a surfactant at a concentration between 0.1% and 10% v/v in a carrier agent.
